(19) United States
(12) Patent Application Publication
(10) Pub. No.: US 2007/0274967 A1
(43) Pub. Date: Nov. 29, 2007

(54) Compositions and methods of treating burn victims using stem cells

(76) Inventor: Calvin Cao, Tampa, FL (US)

Correspondence Address: Calvin Cao, Suite 901, 2203 North Lois Ave, Tampa, FL, 33607, US

(21) Appl. No.: 11/732,985

(22) Filed: Apr. 5, 2007

Related U.S. Application Data
(60) Provisional application No. 60/803,086, filed on May 24, 2006.

(51) Int. Cl. A61K 35/36

(52) U.S. Cl. 424/93.7

(57) ABSTRACT

The present disclosure relates to a method for treating severe burns using a preparation of tissue-derived material, which may comprise stem cells. The method may include systemic administration of stem cells. The method may also include administration of topical preparations of stem cells or tissue extract.

This application claims the benefit of U.S. Provisional Patent, Application No. 60/803,086, filed May 26, 2006, which is incorporated herein by reference.

### FIELD OF THE INVENTION

The present invention relates generally to the field of burn wound repair utilizing partially or completely undifferentiated cells with particular emphasis on stem cells such as human embryonic stem cells, cord blood stem cells and placental cells as well as stem cells of other origin both human and non-human. More specifically, the present invention relates to compositions and methods of ensuring successful autoplasty to repair burn wounds.

### BACKGROUND OF THE INVENTION

Burns are an injury to bodily tissue caused by heat, electricity, chemicals or radiation. There are three levels of burns. First-degree burns affect only the epidermis, i.e. the outer layer of skin, and are characterized by redness, pain, and swelling, but no blisters. The typical sunburn is an example. Second-degree burns (partial thickness burns) affect the epidermis and the underlying dermis, and are characterized by blistering, pain, redness and swelling. Second-degree burns are classified as either superficial or deep, with the deep second-degree burn potentially having a white appearance. The most severe burn, the third-degree burn, affects the epidermis, dermis and further underlying tissue, possibly including the fascia, muscle, or bone. Third-degree burns may be charred or white, often have a leathery appearance, may have purple fluid instead of clear fluid as in the second-degree burn, and a lack of pain due to the nerve endings having been burned away.

Over two million burn injuries are reported per year, with about a quarter of those receiving treatment by a medical professional. First-degree burns are relatively simple to treat, and in many cases are not reported. Cool water is applied to the burn for a period of time and the burn is covered with a sterile dressing to protect it. The burn will in almost all cases heal spontaneously and with a very low likelihood of scarring.

Second-degree burns are more serious and are more commonly reported than first-degree burns. Superficial second-degree burns are at times treatable without a doctor. If the skin is unbroken, a similar procedure to the first-degree burn may be used, taking care not to puncture any blister that may be forming. The more severe second-degree burns, those of the deep variety, should be treated by a medical professional. They typically do not require any surgical intervention but in extreme cases may require grafting. Scarring is common with second-degree burns, particularly after healing of the deep variety.

Third-degree burns, given their severity, must be treated by a medical professional. Hospitalization is common for these burns, and they are typically treated by debridement and surgical skin grafting or transplantation. Significant difficulties arise in treatment of such severe burns, including the failure of the skin graft to properly develop and infections of the wound bed before the repair is complete.

Stem cells are relatively undifferentiated cells that retain the ability to divide and cycle throughout postnatal life to provide cells that can become specialized and take the place of those that die or are lost. Stem cells represent a fundamental building block whose progeny eventually result in the estimated 220 types of specialized cells and tissues in a human. Stem cells are present in human embryos (in blastocysts), the placental complex, blood found in the umbilical cord at birth, bone marrow as well as numerous other tissues.

Recently the source of pluripotent hemopoietic stem cells has expanded to include autologous and allogenic peripheral blood, cord blood and blastocysts. Pluripotent stem cells are generally believed to have the ability to develop into essentially every cell, tissue and organ system found in the human body. For example, the term pluripotent is generally understood to describe stem cells that can develop into cells of all three embryonic germ layers—the mesoderm, endoderm and ectoderm.

Currently stem cell suspension preparations are being used by Stem Cell Therapy International in Ukraine and Mexico to treat a wide variety of afflictions including cardiovascular disease, connective tissue disease, respiratory disease, digestive tract disorders, liver disease, kidney and urinary tract diseases, diabetes, diseases of the nervous system, the consequences of cerebral stroke, blood disorders, ocular disease as well as numerous other diseases, disorders and maladies.

Due to the severity of third-degree burns and the problems associated with the present treatment options for treating such burns, the development of a stem cell based therapy to treat severe burns while reducing or overcoming such problems is highly desirable.

### SUMMARY OF THE INVENTION

The present invention involves preconditioning the wound bed of a severe exterior burn such as a third degree burn after removal of the overlaying injured skin utilizing stem cell preparations so that it readily and relatively quickly accepts repair by autoplasty. It may also involve a biological debridement of a burn to remove necrotic suppurative masses by applying and then removing stem cell preparations from the wound bed. It further may involve preconditioning the wound bed by the intravenous administration of a stem cell preparation to the patient before the autologous skin graft is begun. In addition it may involve treating the debrided wound bed with another stem cell preparation which comprises embryonic fibroblasts, placental tissue or a combination of both. In a preferred embodiment the debridement is achieved using embryonic epithelial cells and in a particularly preferred embodiment these cells are provided by chorion or amniotic sac containing preparations. It is also preferred to use umbilical cord blood as the source of the intravenously administered stem cells, particularly cord blood which has been matched to the patient with regard to blood group and Rh-factor. It is also preferred to use a composition for intravenous administration which has a concentration of between about 108 and 10¹⁰ cells per ml, especially between about 0.2 and 4×109 cells per ml. It is particularly preferred to use mono-nucleated cells, especially embryonic or fetal hemopoietic cells in the intravenously administered preparation, especially if such cells were obtained from peripheral blood or bone marrow. It is also preferred to utilize embryonic fibroblasts, particularly those that have been cultured in vitro, to treat the wound bed after the biological debridement and before the autoplasty. In a particularly preferred embodiment one or more of the cord blood stem cells, the embryonic epithelial cells, the chorion tissue, the amniotic sac tissue or the placental material is provided as a cryopreserved material particularly a material prepared in accordance with the teachings of Ukrainian Patent Application No. UA 60238 or Russian Federation Patent Application No. RU2233589. In a particularly preferred embodiment the intravenous administration of the stem cell preparation is begun before the application of stem cell preparations directly to the burn wound and is continued until complete engraftment of the skin graft.

The treatment of the burn wound bed with the stem cell containing biological preparation by direct and systemic administration serves to stabilize the hematological and biochemical indices of the burn victim as well as stimulating his reparative and regenerative processes. This in turn reduces the instances and severity of infectious and inflammatory processes and reduces or eliminates the manifestations of secondary complications. Thus the character of the wound healing is changed from that which is typically observed and autoskin engraftment is more readily achieved and autoimmune reactions are reduced or eliminated.

### DETAILED DESCRIPTION OF THE INVENTION

While the present invention is capable of embodiment in various forms, hereinafter is described an embodiment with the understanding that the present disclosure is to be considered as an exemplification of the invention, and is not intended to limit the invention to the specific embodiment illustrated. Headings are provided for convenience only and are not to be construed to limit the invention in any way. Embodiments illustrated under any heading may be combined with embodiments illustrated under any other heading.

The use of the terms “a” and “an” and “the” and similar referents in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the invention.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of cardiology and pharmaceutical sciences or the art relevant to the range or element at issue. The amount of broadening from the strict numerical boundary depends upon many factors. For example, some of the factors to be considered may include the criticality of the element and/or the effect a given amount of variation will have on the performance of the claimed subject matter, as well as other considerations known to those of skill in the art. Thus, as a general matter, “about” or “approximately” broaden the numerical value, yet cannot be given a precise limit. For example, in some cases, “about” or “approximately” may mean ±5%, or +10%, or +20%, or +30% depending on the relevant technology. Also, the disclosure of ranges is intended as a continuous range including every value between the minimum and maximum values.

As used herein “pharmaceutical composition” means a composition comprising a preparation which may or may not contain cells, including stem cells, and at least one ingredient which is not an active ingredient whereby the composition can be safely and effectively used as a product to obtain or achieve a desired outcome. The term “pharmaceutical composition” as used herein further means compositions which result from the combination of individual components which are themselves pharmaceutically acceptable. For example, where intravenous administration is foreseen, the components are suitable or acceptable (in both quality and quantity) for intravenous administration.

The stem cell preparations may be prepared and/or preserved according to Ukrainian patent application No. UA 60238 and Russian Federation patent application No. RU2233589, further utilizing Ukrainian patent application No. UA 63844, the disclosures of which are hereby incorporated by reference in their entireties as if fully set forth herein. Preparations from placenta may be obtained according to Ukrainian patent application No. UA 59096, the disclosure of which is also hereby incorporated by reference in its entirety as if fully set forth herein.

In one embodiment of the method, the treatment is started with introduction of a stem cell preparation by IV drip, which maybe maintained for the thirty day course of treatment using saline solution with cell concentration of between 0.2 and 4 billion cells per ml. This systemic introduction of stem cells aids in the regeneration of tissues in the injured patient and supports the other steps in some embodiments of the treatment method. In another embodiment the stem cell preparation is a cord blood stem cell preparation. In another embodiment, the stem cells are embryonic or fetal hematopoietic cells. In a further embodiment, the stem cells are obtained from bone marrow. In another embodiment, the stem cells are obtained from peripheral blood.

In one preferred embodiment after the injured skin of a severe burn wound is removed the wound bed is treated with cryopreserved multicellular chorion tissue in which the viability and biological activity of the natural components of this tissue have been maintained. The application of this material effects a closing of the wound surface preventing the loss of moisture, proteins, electrolytes and other biological materials needed for this underlying tissue to successfully participate in autologous skin engraftment as well as protecting this tissue from the penetration of biological flora. It also stimulates the formation of fibronectin granulosa. This leads to a decrease in the efforts needed to prepare the wound bed for autoplasty as well as increasing tolerance to the skin graft resulting in more readily obtaining full engraftment of the autograft.

In one embodiment, on the fourth day of treatment the burned skin is removed. A topical preparation, such as a cream, paste, spray or other suitable topical vehicle of administration, containing stem cells is then immediately laid on the wound surface. In one embodiment the preparation comprises embryonic epithelial cells. In another embodiment the preparation comprises chorion containing stem cells. In a further embodiment the preparation comprises amniotic sac containing stem cells. In still another embodiment the preparation comprises some combination of embryonic epithelial cells, chorion stem cells, and/or stem cells obtained from amniotic sac. Such cells may be, for example, undifferentiated stem cells, progenitor cells, partially differentiated cells-precursors, fully differentiated cells, or a mixture thereof. It will be appreciated by one of skill in the art that the stem cells may be delivered in a preparation utilizing any one of a variety of pharmaceutically acceptable carriers, for example a water soluble base of oil in water or water in oil, fatty paste bases such as a zinc oxide paste, aqueous gel type pastes such as carboxymethylcellulose sodium paste, ointment bases such as white petrolatum, hydrophilic petrolatum, anhydrous lanolin, lanolin, polyethylene glycol ointment, gel based on compounds such as tragacanth, sodium alginate, gelatin, methylcellulose, carbomer, polyvinyl alcohols. See generally Remington: The Science and Practice of Pharmacy (20th Edition, 2000). The invention is not limited to topical delivery compositions utilizing these compounds, as it will be readily apparent to one skilled in the art that any other suitable compounds for topical delivery may be used and still be within the scope of the present invention.

In one embodiment of the treatment program, on the seventh day of treatment the first preparation is removed. In one embodiment, after removal of the first preparation a paste, cream, or other suitable topical vehicle of administration comprising stem cells from placental tissue is applied to cover the wound surface. Such stem cells may be, for example, undifferentiated stem cells, progenitor cells, partially differentiated cells-precursors, fully differentiated cells, or a mixture thereof. In another embodiment, fibroblasts are laid on the wound surface. In a further embodiment, the fibroblasts are laid on top of the paste or cream comprising placenta tissue. It will be appreciated by one of skill in the art that placenta tissue preparation may be delivered in a topical preparation utilizing any one of a variety of pharmaceutically acceptable carriers, such as a water soluble base of oil in water or water in oil, fatty paste bases such as a zinc oxide paste, aqueous gel type pastes such as carboxymethylcellulose sodium paste, ointment bases such as white petrolatum, hydrophilic petrolatum, anhydrous lanolin, lanolin, polyethylene glycol ointment, gel based on compounds such as tragacanth, sodium alginate, gelatin, methylcellulose, carbomer, polyvinyl alcohols. The fibroblasts may also be delivered in a similar manner. The invention is not limited to topical delivery compositions utilizing only these compounds, as it will be readily apparent to one skilled in the art that any other suitable compounds for topical delivery may be used and still be within the scope of the present invention.

In one embodiment of the treatment program, after the wound surface is covered by the paste and/or the fibroblasts, autoplasty is performed with the graft being laid on top of the paste and/or fibroblasts.

Those skilled in the art will appreciate that numerous other embodiments and modifications are contemplated by the present invention. The above description of embodiments is merely illustrative and not intended to limit the scope of the present invention. The patents, literature, and references cited herein are incorporated by reference in their entireties.

### EXAMPLES

The following Examples are provided for illustrative purposes only and are not to be interpreted as limiting the scope of the present invention in any manner.

### Example 1

A patient with severe burns was treated using an embodiment of the inventive method. Stem cells from cord blood were introduced intravenously in the form of an IV drip. On the second day after the burn injury occurred, a decompression dissection of integuments was performed on the patient.

On the fourth day after the burn injury, the injured skin sites were removed. After removal of the injured skin, a topical preparation containing stem cells, including embryonic epithelial cells and stem cells derived from chorion, was laid on the wound surface.

On the seventh day after the burn injury, said topical preparation was removed and the sites were cleansed of necrotic suppurative masses. The wound sites were then covered with a thin layer of paste containing fragmented placental tissue. Embryonic fibroblasts were also laid upon the wound surface. Autoplasty was then carried out on top of the paste and embryonic fibroblasts.

After 30 days, remarkable results were achieved, as the grafts were accepted well by the patient without autoimmune reaction, and healing appeared to be substantially faster than it would be in a typical autoplasty procedure. Hematologic and biochemical indices of the patient were stable.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles. All patent and literature references are hereby incorporated by reference as if fully set forth herein.

What is claimed is:

1. A method of preconditioning the wound bed of a severe exterior burn to accept a skin graft after removal of the overlaying injured skin comprising treating the burn victim both locally and systemically with stem cell preparations.

2. The method of claim 1 wherein the burn victim is treated with an intravenous administration of a stem cell preparation and the application of a stem cell preparation to the wound bed.

3. A method of debridement of a severe exterior wound after removal of the overlaying injured skin comprising applying to and removing from the wound bed a stem cell preparation

4. The method of claim 3 wherein the stem cell preparation comprises chorion tissue, amniotic sac tissue or a combination of both.

5. A method of preconditioning the wound bed of a severe exterior burn to accept a skin graft after removal of the overlaying injured skin comprising:
a. an intravenous administration of a stem cell preparation;
b. an initial application of a stem cell preparation comprising chorion tissue, amniotic sac tissue or a combination of both to the wound bed followed by its removal; and
c. a subsequent application of a stem cell preparation comprising placental tissue or embryonic fibroblasts to the wound bed.

6. The method of claim 5 wherein the subsequently applied stem cell preparation comprises embryonic fibroblasts which have been cultured in vitro.

7. The method of claim 5 wherein the intravenously administered stem cell preparation comprises umbilical cord blood.

8. The method of claim 7 wherein the cord blood has been matched as to blood group and Rh-factor to that of the burn victim.

9. The method of claim 8 wherein the preparation has a cell concentration of between about 108 and 10¹⁰ cells per ml.

10. The method of claim 9 wherein the preparation has a cell concentration of between about 0.2 and 4×109 cells per ml.

11. The method of claim 5 wherein the intravenous administration is initiated before the initial application of a stem cell preparation to the wound bed.

12. The method of claim 11 wherein the intravenous administration is continued after the subsequent application of a stem cell preparation.

13. The method of claim 5 wherein one or more of the three stem cell preparations is obtained from cryopreserved cells.

14. A method of treating a burn victim in need of treatment, comprising the steps of:
a) providing a composition comprising a plurality of stem cells; and
b) topically administering the composition to at least one burn site of the victim.

15. The method of claim 14, wherein the plurality of stem cells comprises a mixture of undifferentiated stem cells, progenitor cells, partially differentiated cells-precursors, and fully differentiated cells.

16. The method of claim 15, wherein the mixture comprises stem cells and progenitor cells as of epithelial origin.

17. The method of claim 16, wherein the mixture comprises embryonic epithelial cells.

18. The method of claim 15, wherein the mixture comprises cells obtained from chorion.

19. The method of claim 15, wherein the mixture comprises cells obtained from amniotic sac.

20. The method of claim 15, wherein the mixture comprises cells obtained from placenta.

21. The method of claim 14, wherein the composition further comprises cells which were long-term stabilized before resuscitation and application.

22. The method of claim 21, wherein the stabilized cells are cryopreserved cells.

23. The method of claim 21, wherein the stabilized cells are lyophilized cells.

24. The method of claim 21, wherein the stabilized cells are air- or vacuum dried without ice formation.

25. The method of claim 15, wherein the mixture further comprises fibroblasts.

26. The method of claim 25, wherein the fibroblasts are of embryonic origin.

27. The method of claim 16, wherein the mixture further comprises fibroblasts.

28. The method of claim 27, wherein the fibroblasts are of embryonic origin.

29. The method of claim 16, wherein the cells of epithelial origin are laid on the wound region after a decompressive dissection of integuments and removal of the injured skin.

30. The method of claim 18, wherein the chorion cells cover the wound area.

31. The method of claim 19, wherein the cells obtained from amniotic sac cover the wound area.

32. The method of claim 20, wherein the cells obtained from placenta cover the wound area.

33. The method of claim 15, wherein one mixture comprises cells obtained from placenta and another mixture comprises cells obtained from chorion, wherein the cells obtained from placenta cover the wound area after the chorion cells are removed from the wound area.

34. The method of claim 15, wherein one mixture comprises cells obtained from placenta and another mixture comprises cells obtained from amnion, wherein the cells obtained from placenta cover the wound area after the amniotic cells are removed from the wound area.

35. The method of claim 15, wherein the mixture further comprises fibroblasts cover the wound area after a mixture comprising chorion cells is removed from the wound area.

36. The method of claim 15, wherein the mixture further comprises fibroblasts cover the wound area after a mixture comprising amniotic cells is removed from the wound area.

37. The method of claim 14, further comprising the steps of:
a) providing a formulation comprising a plurality of stem cells; and
b) systemically administering the formulation to the burn victim.

38. The method of claim 37, wherein the plurality of stem cells are obtained from umbilical cord.

39. The method of claim 37, wherein the plurality of stem cells are embryonic or fetal hematopoietic cells.

40. The method of claim 37, wherein the plurality of stem cells are obtained from bone marrow.

41. The method of claim 37, wherein the plurality of stem cells are obtained from peripheral blood.

42. The method of claim 37, wherein the plurality of stem cells comprises cells which were long-term stabilized before resuscitation and application.

43. The method of claim 42, wherein the long-term stabilized cells are cryopreserved cells.

44. The method of claim 42, wherein the long-term stabilized cells are lyophilized cells.

45. The method of claim 42, wherein the long-term stabilized cells are air- or vacuum dried without ice formation.

46. The method of claim 37, wherein the stem cells were propagated (e.g., by means of growing in culture).

47. The method of claim 37, wherein formulation comprising a plurality of stem cells is administered intravenously in the amount of 108-10¹⁰ cells.

48. The method of claim 47, wherein the stem cells are mono-nucleated cells.

49. The method of claim 37, wherein formulation comprising a plurality of stem cells is administered intravenously in the amount of 0.2-4×10⁹ cells.

50. The method of claim 49, wherein the stem cells are mono-nucleated cells.

51. A method of treating a burn victim in need of treatment, comprising the steps of:
a) providing a composition comprising non-cellular material extracted from chorion and/or placenta and/or amniotic sac; and
b) topically administering the composition to at least one burn site of the victim.

52. The method of claim 51, wherein the composition was cryopreserved, air- or vacuum dried, or lyophilized prior to application.

53. The method of claim 52, further comprising the steps of:
a) providing a formulation comprising a plurality of stem cells; and
b) systemically administering the formulation to the burn victim.

54. The method of claim 53, wherein the plurality of stem cells are obtained from umbilical cord.

55. The method of claim 53, wherein the plurality of stem cells are embryonic or fetal hematopoietic cells.

56. The method of claim 53, wherein the plurality of stem cells are obtained from bone marrow.

57. The method of claim 53, wherein the plurality of stem cells are obtained from peripheral blood.

58. The method of claim 53, wherein the plurality of stem cells comprises cells which were long-term stabilized before resuscitation and application.

59. The method of claim 58, wherein the long-term stabilized cells are cryopreserved cells.

60. The method of claim 58, wherein the long-term stabilized cells are lyophilized cells.

61. The method of claim 58, wherein the long-term stabilized cells are air- or vacuum dried without ice formation.

62. The method of claim 53, wherein the stem cells were propagated (e.g., by means of growing in culture).

63. The method of claim 53, wherein formulation comprising a plurality of stem cells is administered intravenously in the amount of 108-10¹⁰ cells.

64. The method of claim 63, wherein the stem cells are mono-nucleated cells.

65. The method of claim 53, wherein formulation comprising a plurality of stem cells is administered intravenously in the amount of 0.2-4×10⁹ cells.

66. The method of claim 65, wherein the stem cells are mono-nucleated cells.
